(19) United States
(12) Patent Application Publication
(10) Pub. No.: US 2018/0212257 A1
(43) Pub. Date: Jul. 26, 2018

(54) CONTROL OF SWELLING OF PRIMARY CELLS THROUGH ELECTROLYTE SELECTION

(71) Applicant: Pacesetter, Inc., Santa Clara, CA (US)

(72) Inventor: Timothy R. Marshall, Pickens, SC (US)

(21) Appl. No.: 15/863,444

(22) Filed: Jan. 5, 2018

Related U.S. Application Data
(60) Provisional application No. 62/448,832, filed on Jan. 20, 2017.
(60) Provisional application No. 62/465,524, filed on Mar. 1, 2017.

(51) Int. Cl. H01M 6/50; H01M 6/16; H01M 2/10; A61N 1/378

(52) U.S. Cl. CPC H01M 6/50; H01M 6/164; H01M 2300/0037; A61N 1/378; H01M 2/1022

(57) ABSTRACT

The present systems, i.e. a primary lithium battery, utilize electrolytes that do not produce gases at the lower voltages, allowing increased useable capacity of a battery in a low power implantable medical device.

### PRIORITY

This application claims the benefit of U.S. Provisional Application No. 62/448,832, filed Jan. 20, 2017 and U.S. Provisional Application No. 62/465,524, filed Mar. 1, 2017.

### FIELD

The present systems relate to the design and selection of electrolyte used in batteries for use within implantable medical devices.

### BACKGROUND

Batteries used in implantable medical devices (IMDs), such as cardiac pacemakers and implantable cardioverter defibrillators (ICDs), are required to meet high quality and performance specifications and reliability. Such batteries should possess high energy density, high rate capability and long shelf life. Since replacement of the battery of an IMD means that the patient must undergo surgery, batteries for IMDs are designed to have a long service life. Improvement to the reliability, performance, and lifetime of lithium anode batteries is highly desirable.

New materials and compositions have continually been investigated to improve the capabilities of the different components of high energy density batteries. In particular, the size, shape, surface area, and electrochemical properties of the electrodes (anode and cathode) have been examined to improve the life and efficiency of primary batteries.

In addition to the electrodes, the electrolyte composition employed in a primary battery can affect the life and efficiency of the battery. In primary lithium batteries, the electrolytes consist of lithium salt/salts dissolved in a solvent or mixture of solvents. Typical solvents include propylene carbonate (PC) or ethylene carbonate (EC). Propylene carbonate is used in many battery electrolytes due to its high dielectric constant (64.4), low cost, and wide temperature range (melting point −49° C., boiling point. 241° C.). Hermetic batteries with electrolytes having propylene carbonate as a solvent or co-solvent begin producing propene gas (Scheme 1) at low voltages ˜2.2V, and the reaction increases with a decrease in voltage. As propene is produced the cell pressure increases and causes the case to swell. Ethylene carbonate is also a common battery solvent and will break down in a similar manner as propylene carbonate at low voltages causing the production of ethene gas.

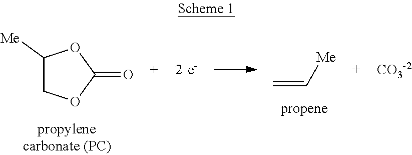

The lithium salt can also play a role in the case swelling due to gas evolution. For example, lithium hexafluoroarsenate (LiAsF₆), commonly used in SVO batteries can break down forming arsenic pentafluoride gas (AsF₅) (Scheme 2).

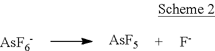

In a commercial cell designed for consumer applications, vents can allow these gases to escape, but in a hermetically sealed medical battery the case builds internal pressure. As a result the case will begin to swell which puts pressure on the adjacent electronics, and potentially causing the IMD to fail. Therefore, a battery end of service (EOS) point is typically chosen to be in the 2.4-2.6 V range to minimize these potential problems, at the cost of additional usable battery capacity.

Processes have been developed in an attempt to address swelling in alkali metal/transition metal oxide cells. U.S. Pat. No. 6,171,729 to Gan, incorporated herein by reference, describes a process to rebalance the anode to cathode (A/C) ratio based on the equivalents of lithium required to completely discharge one equivalent of SVO in a solvent composed of 50:50, by volume, of propylene carbonate:1,2-dimethoxyethane (PC:DME). This process is alleged to reduce the amount of PC decomposition, however, cells begin to swell at a cell potential of ˜2.5 V.

U.S. Pat. Nos. 6,498,951 and 6,899,951, both to Larson et al., which are incorporated herein by reference, describe a strategy where the swelling of the flat liquid electrolyte battery is directed away from the electronics with a particular layout of the internal IMD components and use of “dead space” within the IMD.

It would be advantageous to deplete the batteries to lower voltage levels (1.5-2.0V), increasing the usable capacity of the battery. In FIG. 1, the SVO cell, with an end of service (EOS) at 65-70% depth of discharge (DOD), could potentially be used to 90% or greater. This would result in almost a 20% increase to the life of the battery, if the cell swelling could be eliminated or minimized. In FIG. 2, the CFx (fiber) cell, with an EOS at 80-90% DOD, could potentially be used to 90% or greater, if the cell swelling could be eliminated or minimized. Currently produced medical batteries swell in the <2.2V range due to gases being produced due to the reduction of the battery solvent or salt.

The present disclosure describes electrolytes that do not produce gases at the lower voltages, allowing increased useable capacity of the battery in a low power IMD.

### SUMMARY OF THE INVENTION

Embodiments of a battery electrolyte system are described herein.

The present disclosure provides an improved battery system that can be used to 1.5V without causing the hermetically sealed case of the IMD to swell due to gas formation. The resulting additional battery capacity available down to 1.5V will allow low power implantable medical devices to have longer device lives or reduced volumes without the concern of battery swelling.

Embodiments of a primary lithium battery and electrolyte systems are described herein.

One embodiment of the disclosure is a medium (about 1 mA to about 20 mA) to high rate (about 1 A to about 5 A) hermetically sealed lithium primary battery, comprising
a cathode composed from the group consisting of:
   a) SVO,
   b) CFₓ,
   c) SVOP,
   d) MnO2, and
   e) a combination of any of a, b, c or d, either blended or in distinct layers:
an anode: and
an electrolyte comprising:
   a) a solvent comprising gamma-butyrolactone (GBL), and
   b) a salt comprising about 0.75M to about 2M of a lithium fluoroalkyl sulfonyl imide or about 0.75M to about 2M of a lithium fluorosulfonyl imide; or a mixture thereof,
   c) wherein the electrolyte has a conductivity >10 mSiemen at 20° C.

The present disclosure provides improved electrolyte systems that do not produce gases at lower voltages, allowing increased useable capacity of the battery in a low power implantable medical device such as an implantable cardioverter defibrillator (ICD), pacemaker, or leadless pacemaker. In one embodiment, the present disclosure provides an implantable medical device, such as a leadless pacemaker that includes a medium to high rate hermetically sealed lithium primary battery as described herein.

### BRIEF DESCRIPTION OF THE FIGURES

The accompanying figure, which is incorporated herein and form part of the specification, illustrate a known system and electrolyte. Together with the detailed description, the figure further serve to explain the principles of, and to enable a person skilled in the relevant art(s) to make and use, the batteries and systems presented herein.

FIG. 1 illustrates a discharge curve of a typical SVO cell utilizing a conventional electrolyte system.

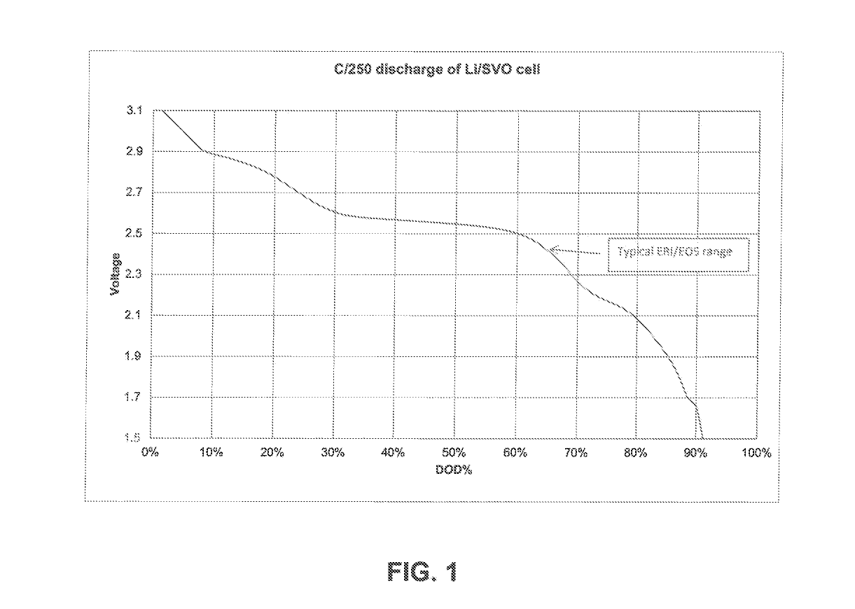

FIG. 2 illustrates a discharge curve of a typical CFx (fiber) cell utilizing a conventional electrolyte system.

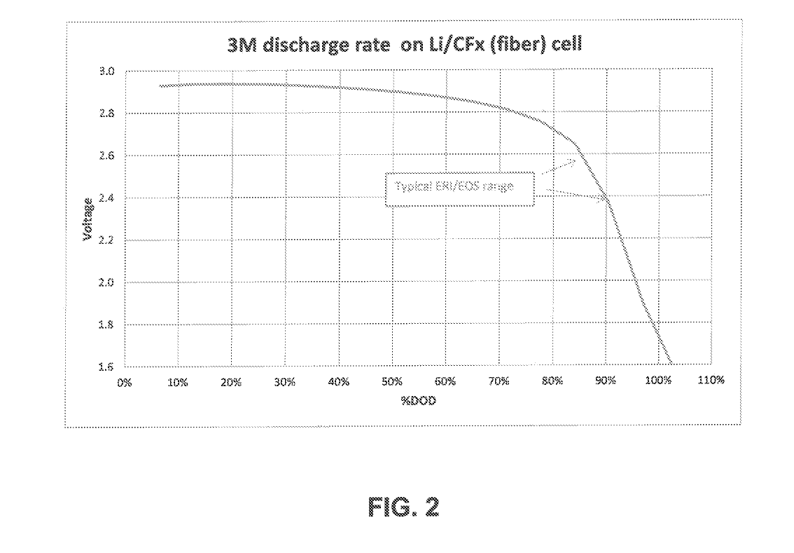

### DETAILED DESCRIPTION

The following detailed description of the batteries and electrolyte systems describe exemplary embodiments. Other embodiments are possible, and modifications may be made to the embodiments within the spirit and scope of the batteries and systems presented herein. Therefore, the following detailed description is not meant to limit the batteries and electrolyte systems described herein. Rather, the scope of these batteries and electrolyte systems is defined by the appended claims.

The lithium primary battery and electrolyte embodiments described herein are useful in the environment of an IMD. Examples of batteries, electrolyte systems, and IMDs can be found in U.S. Pat. Nos.; 5,154,992; 5,895,733; 6,130,005; 5,955,218; 6,926,991; and 7,651,647, each of which is incorporated herein by reference. The lithium primary battery and electrolyte embodiments described herein are particularly useful in the environment of leadless pacemakers. Examples of leadless pacemaker can be found in U.S. Pat. Nos. 9,265,436 and 9,358,400, each of which is incorporated herein by reference.

An IMD, such as those described in the patents identified above, requires a power source in order to operate. A primary lithium battery comprising an anode, a cathode and an electrolyte solution is used to provide a high current output power source. One aspect of the disclosure describes batteries having an electrolyte system that does not cause swelling due to gas evolution at lower voltages.

In an embodiment of the disclosure, the medium to high rate hermetically sealed lithium primary battery, comprises
a cathode composed from the group consisting of:
   a) SVO,
   b) CFₓ,
   c) SVOP,
   d) MnO2, and
   e) a combination of any of a, b, c or d, either blended or in distinct layers;
an anode; and
an electrolyte comprising:
   a) a solvent comprising gamma-butyrolactone (GBL) and
   b) a salt comprising about 0.75M to about 2M of a lithium fluoroalkyl sulfonyl imide or about 0.75M to about 2M of a lithium fluorosulfonyl imide; or a mixture thereof,
   c) wherein the electrolyte has a conductivity >10 mSiemen at 20° C.

In one embodiment, the cathode is a combination of SVO and CFₓ, either blended or in distinct layers.

In one embodiment, the cathode is a blended combination of SVO and CFₓ.

In one embodiment, the cathode is a blended combination of SVO and CFₓ wherein the CFₓ is about 5% to about 95% by mass of the blended combination.

In one embodiment, the lithium primary battery is usable to about 1.5V without swelling due to gassing.

In the present disclosure, the term “silver vanadium oxide” or “SVO” refers to a family of silver vanadium oxide cathodes having the general formula AgₓV₂O_{y} in any one of its many phases, i.e. β-phase silver vanadium oxide having in the general formula x=0.35 and y=5.8; γ-phase silver vanadium oxide having in the general formula x=0.74 and y=5.37; and ε-phase silver vanadium oxide having in the general formula x=1.0 and y=5.5, and combination and mixtures of phases thereof.

In the present disclosure, the term “carbon monofluoride” or “CFₓ” refers to a family of carbon fluoride cathodes having the general formula CFₓ in any of its forms where in the general formula x varies between about 0.1 to about 1.9 and preferably between about 0.5 to about 1.4.

The preferred solvent for the battery electrolyte is gamma-butyrolactone (GBL) either as a solitary solvent or as a co-solvent blended with 1,2-dimethoxyethane (DME). Gamma-Butyrolactone GBL behaves differently from propylene carbonate. Gamma-Butyrolactone (GBL) does not form a gas at low voltages but forms a salt, causing a net volume reduction (Scheme 3). See, Aurbach, D., “Identification of Surface films Formed on Lithium surfaces in γ-Butyrolactone solutions” J. Electrochem. Soc. 136:1606-1610 (June 1989).

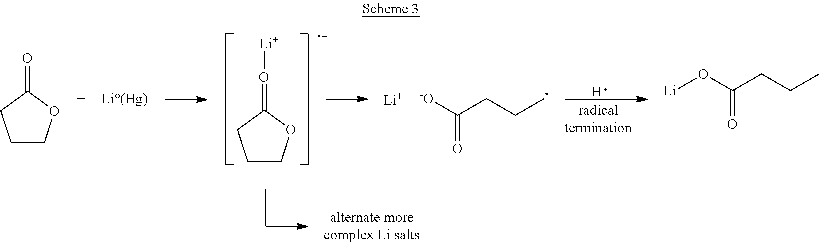

In one embodiment of the disclosure the solvent for the lithium primary battery electrolyte comprises gamma-butyrolactone.

In one embodiment of the disclosure the solvent for the lithium primary battery electrolyte comprises a mixture of gamma-butyrolactone and 1,2-dimethoxyethane

In one embodiment of the disclosure the solvent for the primary battery electrolyte comprises about 20% to about 99.9% gamma-butyrolactone and about 0.1% to about 80% 1,2-dimethoxyethane.

In one embodiment of the disclosure the solvent for the primary battery electrolyte comprises about 50% to about 99.9% gamma-butyrolactone and about 0.1% to about 50% 1,2-dimethoxyethane.

In one embodiment of the disclosure the solvent for the primary battery electrolyte comprises about 50% gamma-butyrolactone and about 50% 1,2-dimethoxyethane.

A suitable electrolyte for a primary electrochemical cell has an inorganic, ionically conductive salt dissolved in a nonaqueous solvent. More preferably, the electrolyte includes an ionizable alkali metal salt dissolved in a mixture of aprotic organic solvents comprising a low viscosity solvent and a high permittivity solvent. In the case of an anode comprising lithium, the alkali metal salt is lithium based. Known lithium salts useful as vehicles for transport of lithium ions from the anode to the cathode include LiBF₄, LiN(SO₂CF₃)₂, LiN(SO₂F₂)₂, and mixtures thereof. The salt used in the electrolyte also requires stability at low voltages (e.g. 1.5-2.0V). The preferred salts include lithium fluoroalkyl sulfonyl imides or a lithium fluorosulfonyl imide.

Specifically, lithium bis(trifluoromethanesulfonyl) imide formula I (LiTFSi) ((CF₃S₀₂)₂NLi) is much more stable than LiAsF₆ in that the fluorine-carbon bond in LiTFSi is stronger than the As—F bond in LiAsF_{6.}

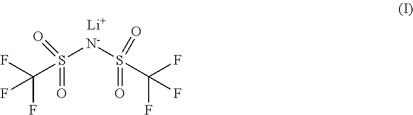 [SMILES: [Li+] ; O=S(=O)([N-]S(=O)(=O)C(F)(F)F)C(F)(F)F]

An additional salt that shows similar stability to LiTFSi is lithium bis(fluorosulfonyl) imide, formula II (LiFSi) ((F₂SO₂)₂NLi). The difference between the two salts is that LiTFSi has a trifluoromethyl group attached to the sulfur atom where LiFSi has a fluorine attached to the sulfur atom.

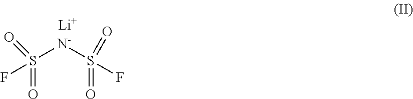

In one embodiment of the disclosure the salt for the primary battery electrolyte is lithium bis(trifluoromethanesulfonyl) imide, lithium bis(fluorosulfonyl)imide, or a mixture thereof.

In one embodiment, the salt for the primary battery electrolyte comprises about 0.75M to about 2M lithium bis(trifluoromethanesulfonyl) imide or lithium bis(fluorosulfonyl) imide.

In one embodiment, the salt for the primary battery electrolyte comprises about 0.75M to about 2M lithium bis(trifluoromethanesulfonyl) imide.

In one embodiment, the salt for the primary battery electrolyte comprises about 0.75M to about 2M lithium bis(fluorosulfonyl) imide.

In one embodiment, the salt for the primary battery electrolyte comprises about 1.0M to about 1.5M lithium bis(trifluoromethanesulfonyl) imide.

In one embodiment, the salt for the primary battery electrolyte comprises about 1.0M to about 1.5M lithium bis(fluorosulfonyl) imide.

In one embodiment, the salt for the primary battery electrolyte comprises about 1.2M to about 1.5M lithium bis(trifluoromethanesulfonyl) imide.

In one embodiment, the salt for the primary battery electrolyte comprises about 1.2M to about 1.5M lithium bis(fluorosulfonyl) imide.

An additional aspect of the disclosure is directed to the battery electrolyte itself. The electrolyte comprises
a) a solvent comprising gamma-butyrolactone (GBL) and
b) a salt comprising about 0.75M to about 2M of a lithium fluoroalkyl sulfonyl imide or a lithium fluorosulfonyl imide; or a mixture thereof.

The electrolyte solution typically has a conductivity of greater than 10 mSiemen at 20° C. Conductivity is measured using a conductivity probe.

In one embodiment, the solvent further comprises 1,2-dimethoxyethane.

In one embodiment, the solvent comprises about 20% to about 99.9% gamma-butyrolactone and about 0.1% to about 80% 1,2-dimethoxyethane.

In one embodiment, the solvent comprises about 50% to about 99.9% gamma-butyrolactone and about 0.1% to about 50% 1,2-dimethoxyethane

In one embodiment, the solvent comprises about 50% gamma-butyrolactone and about 50% 1,2-dimethoxyethane.

In one embodiment, the salt comprises a salt selected from lithium bis(trifluoromethanesulfonyl) imide, lithium bis(fluorosulfonyl) imide, or a mixture thereof.

In one embodiment, the salt comprises about 0.75M to about 2M lithium bis(trifluoromethanesulfonyl) imide or lithium bis(fluorosulfonyl) imide.

In one embodiment, the salt comprises about 0.75M to about 2M lithium bis(trifluoromethanesulfonyl) imide.

In one embodiment, the salt comprises about 0.75M to about 2M lithium bis(fluorosulfonyl) imide.

In one embodiment, the salt comprises about 1.0M to about 1.5M lithium bis(trifluoromethanesulfonyl) imide.

In one embodiment, the salt comprises about 1.0M to about 1.5M lithium bis(fluorosulfonyl) imide.

In one embodiment, the salt comprises about 1.2M to about 1.5M lithium bis(trifluoromethanesulfonyl) imide.

In one embodiment, the salt comprises about 1.2M to about 1.5M lithium bis(fluorosulfonyl) imide.

Another aspect of the disclosure is directed to a leadless pacemaker comprising a medium to high rate hermetically sealed lithium primary battery, comprising
a cathode composed from the group consisting of:
   a) SVO,
   b) CFₓ,
   c) SVOP,
   d) MnO2, and
   e) a combination of any of a, b, c or d, either blended or in distinct layers;
an anode, and
an electrolyte comprising;
   a) a solvent comprising 20-100% gamma-butyrolactone (GBL) and and 0-80% DME
   b) a salt comprising about 0.75M to about 2M of a lithium fluoroalkyl sulfonyl imide or about 0.75M to about 2M of a lithium fluorosulfonyl imide; or a mixture thereof.
   c) wherein the electrolyte has a conductivity >10 mSiemen at 20° C.

Another aspect of the disclosure is related to implantable medical devices that include a lithium primary battery described in the any of the preceding embodiments or aspects.

In the present disclosure, the term “leadless pacemaker” refers to a leadless intra-cardiac medical devices

In the present disclosure, the term “leadless” generally refers to an absence of electrically-conductive leads that traverse vessels outside of the intra-cardiac space.

In the present disclosure, the term “intra-cardiac space” generally means, entirely within the heart and associated vessels.

A leadless pacemaker (LPM) is typically characterized by the following features: it is devoid of leads that pass out of the heart to another component, such as a pacemaker can located outside of the heart; it includes electrodes that are affixed directly to the can of the device and/or near the can; the entire device is attached to the heart; and the device is capable of pacing and sensing in the chamber of the heart where it is implanted. It can be appreciated, however, that an LPM needs to be compact enough to fit within the heart. At the same time, the LPM requires a power source to operate. Accordingly, the pacer module includes a battery contained therein. Typically, the LPM has a housing having a battery that may take up as much as 75% of the internal volume of the housing. It is therefore desirable to minimize the battery size so that the LPM itself can be reduced in size so as not to adversely impact proper heart function. Leadless pacemakers having a battery as described herein are advantageous due to the reduced swelling compared to other hermetically sealed batteries, as well as the longer battery life. Leadless pacemakers are described in U.S. Pat. Nos. 9,265,436 and 9,358,400, each of which is incorporated herein by reference.

The electrolyte solution can be incorporated into a primary or secondary electrochemical cell, as is well known to those of ordinary skill in the art. In that respect, an electrolyte solution of the disclosure can be used, for example, in a nonaqueous electrochemical cell as described in U.S. Pat. No. 4,830,940 to Keister et al., incorporated herein by reference. The electrochemical cell contains an anode of a metal selected from Group IA of the Periodic Table of Elements, including lithium, sodium, potassium, etc., preferably lithium, and their alloys and intermetallic compounds, for example Li—Si, Li—Ai, Li—B and Li—Si—B alloys and intermetallic compounds. The form of the anode may vary, but typically the anode is in the form of a thin sheet or foil of the anode metal, and a current collector having an extended tab or lead affixed to the anode sheet or foil.

The fabrication process for a battery electrode, especially the cathode in case of a primary battery, is critical to achieve these goals. In the fabrication of components for such batteries, electrodes, including anodes and cathodes, are fabricated, at least in part, from electrode active blanks and plates formed from tapes. Various processes and materials are employed to form cathodes for IMD batteries.

### Cathodes Useful in Batteries of the Disclosure

Various processes have been developed to produce battery electrodes, including press powder, tape casting, extrusion, and calender sheeting. U.S. Pat. No. 4,556,618 to Shia, incorporated herein by reference, describes preparation process for preparing of battery cathodes. The process comprises mixing an electrode active material; a conductive carbon additive, such as acetylene black and/or graphite; and polytetrafluoroethylene (PTFE). The process employs high shear mixing to cause the PTFE to fibrillate. The mixture is then wet with a non-polymeric pore-former to make the mixture more pliable and to create micropores in the electrode when the solution is removed by evaporation. The wet mixture is then sequentially extruded, calendered, or pressed to flatten the mixture to a thin sheet and rolled up and folded and pressed out again. Shia teaches that high PTFE levels in an electrode result in higher voltage losses for that electrode, and that a lower level of PTFE binder provides more active material in the electrode and results in electrodes with higher capacity per unit volume.

U.S. Pat. Nos. 5,435,874 and 5,571,640, both to Takeuchi et al., which are incorporated herein by reference, disclose a process for manufacturing a free-standing sheet of cathode material that are typically used in lithium anode cells. The process involves first adjusting the particle size of the cathode active material, followed by mixing with binder and additives suspended in a solvent to form a paste. The paste is fed into a series of roll mills which calender the paste into a sheet form. Alternatively, the paste is first pelletized before being calendered. The resulting cathode sheet material is dried and punched into blanks that are subsequently contacted to a current collector to form an electrode. The pelletized cathode active material, is performed by two to four calender mills which serve to sequentially press the active admixture into a tape of a free-standing sheet having a thickness in the range of about 0.004 inches to about 0.020 inches.

U.S. Pat. Nos. 6,174,622 and 6,582,545 both to Thiebolt, III, et al., incorporated herein by references, disclose a process for forming blanks or plates of electrode active material. A first tape or pellet-shaped structure having a first thickness is provided. It is subsequently calendered in a secondary rolling step, substantially orthogonal to the direction at which the first calendering step occurred or at a second direction, opposite the first rolling step. This secondary rolling step provides a second tape or pellet-shaped structure with a second thickness less than the first thickness of the first tape.

U.S. Pat. No. 7,572,551 to Panzer et al. describes a method for making an electrode tape, which comprises blending an electrode active material, a conductive diluent, a binder and a lubricant to form an electrode active mixture; adjusting the solids content of the electrode active mixture to form a filter cake; crumbling the filter cake into particles of electrode active mixture, and performing at least a primary calendering of the particles of the electrode active mixture at a calendering station by applying a compacting force to forcibly move the particles directly into a nip zone of adjacent calendering rolls to compact the particles of the electrode active mixture in the nip zone.

The cathode in some primary lithium batteries currently used in implantable medical devices can consist of carbon monofluoride (CFₓ), silver vanadium oxide (SVO), manganese dioxide (MnO₂) or blends of these materials. These chemistries are capable of supplying milli-ampere to multi-ampere level currents to operate the device. Current power requirements in medical devices require the voltage to be greater than 2.1V for operation and require device replacement before the battery is fully depleted. Additionally, as the battery is depleted and voltage decreases in the battery, electrolyte components begin reducing (breaking down) and producing gaseous byproducts.

U.S. Pat. Nos. 4,310,609 and 4,391,729, both to Liang et al., incorporated herein by reference, disclose the preparation of silver vanadium oxide (SVO) by a thermal decomposition of ammonium vanadate to vanadium pentoxide followed by the addition of AgNO₃. This process resulted in SVO after a final heat treatment step of about 360° C. U.S. Pat. No. 5,498,494 to Takeuchi et al., incorporated herein by reference, describes the preparation of SVO from Ag₂O and V₂O₅ by a chemical addition reaction. U.S. Pat. No. 5,221,453 to Crespi., incorporated herein by reference, discloses the preparation of silver vanadium oxide from AgVO₃ and V₂O₅ or Ag₂O and V₂O₅ by a chemical addition reaction in a temperature range of about 300° C. to about 700° C.

U.S. Pat. No. 6,017,656 to Crespi, U.S. Pat. No. 5,180,642 to Weiss, U.S. Pat. No. 6,783,888 to Gan et al., and U.S. Patent Application Publication No. 2007/0178381 to Howard et al., each of which is incorporated herein by reference, disclose the preparation of hybrid cathodes containing a mixture of silver vanadium oxide (SVO) and carbon monofluoride (CFx).

U.S. Pat. Nos. 6,551,747 and 6,783,888, both to Gan, incorporated herein by reference, describe a sandwiched cathode design for use in a high rate electrochemical cell. The sandwich cathode is composed of a first cathode active material of a relatively high energy density but of a relatively low rate capability, such as CFx, Ag₂O₂, and SVO, sandwiched between two layers of current collector.

### Anodes Useful in Batteries of the Disclosure

U.S. Pat. No. 4,271,242 to Toyoguchi et. al. incorporated herein by reference, discloses the use of fluorinated carbon materials obtained by fluorinating carbon having a lattice constant of 3.40-3.50 Å in its (002) plane to prepare a Li/CFx battery. The carbon is selected from petroleum cokes and coal cokes and the resulting battery has excellent discharge and shelf life characteristics. The fluorinated carbon materials described in this patent are generally accepted as the industry standard for lithium batteries employing fluorinated carbon cathodes and such materials are widely used in commercial battery production. Fluorinated petroleum coke is the most commonly used form of fluorinated carbon for Li/CFx cells and this material is described in numerous patents relating to battery construction and operation in the field of implantable medical use. Carbon monofluoride, often referred to as carbon fluoride, polycarbon monofluoride, CFx or graphite fluoride is a solid, structural, non-stoichiometric fluorocarbon of empirical formula CFx, wherein x is 0.01 to 1.9, preferably 0.1 to 1.5, and more preferably 1.1. One commercial form of carbon monofluoride is (CFx)n where 0<x<1.25 (and n is the number of monomer units in the polymer, which can vary widely).

Generally, production of CFx involves an exemplary chemical reaction such as:

F₂+(x+y+z)C→xCF_{1.1}+yC+z(CF_{n≥2})

where x, y, and z are numerical values that may be positive integers or positive rational numbers. In this reaction, fluorine and carbon react to form CF_{1.1.} Unreacted carbon and impurities are by-products of the chemical reaction, which are preferably minimized during production of CFₓ. It is desirable to achieve a weight percentage of fluorine greater than or equal to 61% in CFₓ while reducing impurities. Preferably, greater than or equal to 63% or 65% of fluorine exists in the CFₓ. Purity, crystallinity, and particle shape, particularly of the carbon precursor, are also properties to consider in the selection of carbon monofluoride. This is described in greater detail in U.S. Patent Application Publication No. 2007/0178381 (Howard et al.), incorporated herein by reference.

In order to prevent internal short circuit conditions, the cathode is separated from the Group IA, IIA or IIIB anode material by a suitable separator material. The separator is of electrically insulative material, and the separator material also is chemically unreactive with the anode and cathode active materials and both chemically unreactive with and insoluble in the electrolyte. In addition, the separator material has a degree of porosity sufficient to allow flow there through of the electrolyte during the electrochemical reaction of the cell. Illustrative separator materials include fabrics woven from fluoropolymeric fibers including polyvinylidene fluoride, polyethylenetetrafluoroethylene, and polyethylenechlorotrifluoro-ethylene used either alone or laminated with a fluoropolymeric microporous film, non-woven glass, polypropylene, polyethylene, glass fiber materials, ceramics, a polytetrafluoroethylene membrane commercially available under the designation ZITEX (Chemplast Inc.), a polypropylene membrane commercially available under the designation CELGARD (Celanese Plastic Company, Inc.) and a membrane commercially available under the designation DEXIGLAS (C.H. Dexter, Div., Dexter Corp.).

The preferred form of the primary and the secondary electrochemical cell is a case-negative design wherein the anode/cathode couple is inserted into a conductive metal casing connected to the anode current collector, as is well known to those skilled in the art. A preferred casing material is titanium although stainless steel, mild steel, nickel, nickel-plated mild steel and aluminum are also suitable. The casing header comprises a metallic lid having an opening for the glass-to-metal seal/terminal pin feedthrough for the cathode electrode and an electrolyte fill opening. The cell is thereafter filled with the appropriate electrolyte solution and hermetically sealed such as by close-welding a stainless steel plug over the fill opening, but not limited thereto. The cell of the present invention can also be constructed in a case-positive design.

While various embodiments of the present disclosure have been described above, it should be understood that they have been presented by way of example only, and not limitation. Thus, the breadth and scope of the present disclosure should not be limited by any of the above-described exemplary embodiments, but should be defined only in accordance with the following claims and their equivalents. Additionally, all references cited herein, including journal articles or abstracts, published or corresponding U.S. or foreign patent applications, issued U.S. or foreign patents, or any other references, are each entirely incorporated by reference herein, including all data, tables, figures, and text presented in the cited references.

It must be noted that as used in the present disclosure and in the appended claims, the singular forms “a”, “an”, and “the” include plural reference unless the context clearly dictates otherwise. Illustratively, the term “a salt ” is intended to include one or more salts, including mixtures thereof (e.g., lithium bis(trifluoromethanesulfonyl) imide or lithium bis(fluorosulfonyl)imide, and/or mixtures thereof) and the term “a solvent” is intended to include one or more solvents, including mixtures thereof (e.g. gamma-butyrolactone and 1,2-dimethoxyethane, and/or mixtures thereof).

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

### ABBREVIATIONS

SVO—silver vanadium oxide

CFₓ—carbon monofluoride

SVOP—silver vanadium oxyphosphate

MnO₂—manganese dioxide

GBL—gamma-butyrolactone

DME—1,2-dimethoxyethane

LiTFSi—lithium bis(trifluoromethanesulfonyl) imide

LiFSi—lithium bis(fluorosulfonyl) imide

PC—propylene carbonate

EC—ethylene carbonate

EOS—end of service

DOD—depth of discharge

LPM—Leadless pacemakers

What is claimed is:

1. A medium to high rate hermetically sealed lithium primary battery, comprising
a cathode composed from the group consisting of:
a) SVO,
b) CFₓ,
c) SVOP,
d) MnO2, and
e) a combination of any of a, b, c or d, either blended or in distinct layers:
an anode: and
an electrolyte comprising:
a) a solvent comprising gamma-butyrolactone and
b) a salt comprising about 0.75M to about 2M of a lithium fluoroalkyl sulfonyl imide or about 0.75M to about 2M of a lithium fluorosulfonyl imide; or a mixture thereof.

2. The lithium primary battery of claim 1, wherein the lithium primary battery is usable to about 1.5V without swelling due to gassing.

3. The lithium primary battery of claim 1, wherein the solvent further comprises 1,2-dimethoxyethane.

4. The lithium primary battery of claim 3, wherein the solvent of the electrolyte comprises about 20% to about 99.9% gamma-butyrolactone and about 0.1% to about 80% 1,2-dimethoxyethane.

5. The lithium primary battery of claim 3, wherein the solvent of the electrolyte comprises about 50% to about 99.9% gamma-butyrolactone and about 0.1% to about 50% 1,2-dimethoxyethane.

6. The lithium primary battery of claim 3, wherein the solvent of the electrolyte comprises about 50% gamma-butyrolactone and about 50% 1,2-dimethoxyethane.

7. The lithium primary battery of claim 1, wherein the salt of the electrolyte comprises about 0.75M to about 2M lithium bis(trifluoromethanesulfonyl) imide or lithium bis(fluorosulfonyl)imide.

8. The lithium primary battery of claim 7, wherein the salt of the electrolyte comprises about 0.75M to about 2M lithium bis(trifluoromethanesulfonyl) imide.

9. The lithium primary battery of claim 7, wherein the salt of the electrolyte comprises about 0.75M to about 2M lithium bis(fluorosulfonyl) imide.

10. The lithium primary battery of claim 7, wherein the salt of the electrolyte comprises about 1.0M to about 1.5M lithium bis(trifluoromethanesulfonyl) imide.

11. The lithium primary battery of claim 7, wherein the salt of the electrolyte comprises about 1.0M to about 1.5M lithium bis(fluorosulfonyl) imide.

12. The lithium primary battery of claim 7, wherein the salt of the electrolyte comprises about 1.2M to about 1.5M lithium bis(trifluoromethanesulfonyl) imide.

13. The lithium primary battery of claim 7, wherein the salt of the electrolyte comprises about 1.2M to about 1.5M lithium bis(fluorosulfonyl) imide.

14. The lithium primary battery of claim 1, wherein the electrolyte has a conductivity greater than 10 mSiemen at about 20° C.

15. An electrolyte within a battery, the electrolyte comprising;
a) a solvent comprising gamma-butyrolactone and
b) a salt comprising about 0.75M to about 2M of a lithium fluoroalkyl sulfonyl imide or a lithium fluorosulfonyl imide; or a mixture thereof.

16. The electrolyte of claim 15, wherein the solvent further comprises 1,2-dimethoxyethane.

17. The electrolyte of claim 16, wherein the solvent comprises about 50% to about 99.9% gamma-butyrolactone and about 0.1% to about 50% 1,2-dimethoxyethane.

18. The electrolyte of claim 16, wherein the solvent comprises about 50% gamma-butyrolactone and about 50% 1,2-dimethoxyethane.

19. The electrolyte of claim 16, wherein the solvent comprises 20% to about 99.9% gamma-butyrolactone and about 0.1% to about 80% 1,2-dimethoxyethane.

20. The electrolyte of claim 15, wherein the salt is present at about 0.75M to about 2M lithium bis(trifluoromethanesulfonyl) imide or about 0.75M to about 2M lithium bis(fluorosulfonyl) imide.

21. The electrolyte of claim 20, wherein the salt is present at about 0.75M to about 2M lithium bis(trifluoromethanesulfonyl) imide.

22. The electrolyte of claim 20, wherein the salt is present at about 0.75M to about 2M lithium bis(fluorosulfonyl) imide.

23. The electrolyte of claim 20, wherein the conductivity is greater than 10 mSiemen at about 20° C.

24. The lithium primary battery of claim 1, wherein the end of service point is greater than 70% depth of discharge.

25. The lithium primary battery of claim 1, wherein the end of service point is greater than 80% depth of discharge.

26. The lithium primary battery of claim 1, wherein the end of service point is greater than 90% depth of discharge.

27. A implantable medical device comprising a medium to high rate hermetically sealed lithium primary battery according to claim 1.

28. The implantable medical device of claim 27, wherein the lithium primary battery is usable to about 1.5V without swelling due to gassing.

29. The implantable medical device of claim 28, wherein the implantable medical device is a leadless pacemaker.

30. The lithium primary battery of claim 1, wherein the cathode comprises a combination of SVO and CFₓ, either blended or in distinct layers.

31. The lithium primary battery of claim 30, wherein the cathode comprises a blended combination of SVO and CFₓ.

32. The lithium primary battery of claim 31, wherein the cathode comprises a blended combination of SVO and CFₓ wherein the CFₓ is about 5% to about 95% by mass of the blended combination.
